(19) United States
(12) Patent Application Publication
(10) Pub. No.: US 2019/0175382 A1
(43) Pub. Date: Jun. 13, 2019

(54) HEAD IMMOBILIZATION AID WITH ADJUSTABLE SUPPORTS

(71) Applicant: Tortle Products LLC, Greenwood Village, CO (US)

(72) Inventors: Jane SCOTT, Castle Rock, CO (US); Brendan FITCH, Castle Rock, CO (US); Cameron FITCH, Franktown, CO (US)

(21) Appl. No.: 16/322,997

(22) PCT Filed: Aug. 7, 2017
(86) PCT No.: PCT/US17/45727

§ 371 Date: Feb. 4, 2019

Related U.S. Application Data
(60) Provisional application No. 62/372,190, filed on Aug. 8, 2016.

(51) Int. Cl. A61F 5/37

(52) U.S. Cl. CPC A61F 5/3707; A61G 7/072

(57) ABSTRACT

A head immobilization aid includes immobilization members, a rear panel, at least one strap, and at least one flap. The immobilization members maintain a patient's head in a desired position. The strap secures the head immobilization aid to a patient's head and the flap secures the immobilization members in a desired position. The head immobilization aid may be used in conjunction with an attachment device to secure and orient tubing for continuous positive airway pressure (CPAP) and nasal cannulas to the head immobilization aid.

### BACKGROUND

Head immobilization benefits patients with a wide variety of medical conditions such as respiratory distress or a head, neck, or back injury. Immobilizing the head during the provision of respiratory support, such as through nasal continuous positive airway pressure (CPAP) or a nasal cannula, permits uniform distribution of support to the lungs. Immobilizing the head can help aid in treating a head, neck, or back injury and prevent further injury. Infants and babies benefit from maintaining the head in a midline (neutral) position to reduce the risks of and complications from intraventricular hemorrhage and to promote optimal cerebral blood flow. Patients of all ages benefit from maintaining the head in midline or other desired positions for the effective and unobstructed delivery of medical interventions, such as spinal taps, head shunts, nasal cannulas, nasal CPAP, ventilator support, feeding tubes, and intravenous (IV) fluid support (such as via scalp IVs).

Medical tape placed on the face or scalp, for purposes such as to secure nasal cannulas, feeding tubes, or scalp IVs, can be irritating or damaging to the skin.

The information included in this Background section of the specification, including any references cited herein and any description or discussion thereof, is included for technical reference purposes only and is not to be regarded as subject matter by which the scope of the invention as defined in the claims is to be limited.

### SUMMARY OF THE INVENTION

The technology disclosed herein relates to head immobilization aids. The head immobilization aids may be used to assist desired head positioning; promote uniform distribution of respiratory support to the lungs; treat or prevent a head, neck, or back injury; promote optimal cerebral blood flow; and treat or prevent intraventricular hemorrhage. Head immobilization aids may be used for patients of all ages, e.g., patients with medical issues that require, or would be aided by, head immobilization.

In some embodiments, the head immobilization aid comprises immobilization members, a rear panel, at least one strap (e.g., two straps), and at least one flap (e.g., two flaps). The at least one strap secures the head immobilization aid to the head of a patient. The at least one flap secures the adjacent immobilization member in a desired position. Access to the scalp for placement, removal, and monitoring of scalp IVs is maintained. The immobilization members minimize movement of a patient's head, maintain a patient's head in a desired position, and deflect a patient's moving head back to a desired position. The headwear may also be constructed of a hook-receptive material (e.g., the hook side of hook and loop fastener material components) to reduce the number of attached fasteners, maximize size adjustability, and maximize versatility for attaching medical devices.

In one implementation, a method for using the head immobilization aid, such as to support a patient in a midline position, is provided. The head immobilization aid is applied to the patient's head without disturbing scalp IVs or other medical devices connected to the patient's head, which minimizes stress on the patient. Closing and fastening the straps secures the head immobilization aid to the patient and reduces or prevents the unintentional movement of the headwear. Closing and fastening the flaps secures the immobilization members in a desired position. The immobilization members are positioned on either side of the patient's head and help prevent the head from moving. The patient's head is thereby maintained in a midline position with respect to the spine of the patient when the patient is supine.

In some implementations, the closed straps or flaps provide a surface onto which attachment devices that secure medical devices, such as nasal cannulas and CPAP tubes, to the patient's head can be attached. The attachment devices permit attachment of medical devices without the use of medical tape on a patient's face or elsewhere.

In another implementation, a method for using the head immobilization aid, such as to support a patient lying on the side of its head, while the patient is also lying on its back or it's the side of its body, is provided. The headwear is applied as described above. The immobilization members are positioned towards the back of the patient's head, adjacent to each other, and help to prevent the head from moving. The patient's head is thereby maintained in a desired position, which may be a midline position, when the patient is partially or completely lying on its side.

In another aspect, the invention features a method of treatment using a head immobilization aid.

In another aspect, the invention features a head immobilization aid for use according to any of the methods described herein.

In other implementations, the head immobilization aid is provided as part of a kit that also includes at least one attachment device. A kit allows convenient transport, storage, and laundering of the head immobilization aid and other components.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. A more extensive presentation of features, details, utilities, and advantages of the present invention as defined in the claims is provided in the following written description of various embodiments of the invention and illustrated in the accompanying drawings.

### BRIEF DESCRIPTION OF THE DRAWINGS

FIG. 1 is a front, bottom, left isometric view of a head immobilization aid according to one embodiment with the straps laid open.

FIG. 2 is a front, bottom, left isometric view of the head immobilization aid of FIG. 1 with the straps pulled in and the flaps exposed.

FIG. 3 is a rear, bottom, left isometric view of the head immobilization aid of FIG. 1 with the straps folded in.

FIG. 4 is a front, bottom, left isometric view of the head immobilization aid of FIG. 1 as applied to a patient's head.

FIG. 5 is a front, bottom, left isometric view of the head immobilization aid of FIG. 1 as applied to a patient's head with the straps closed and a flap exposed.

FIG. 6 is a front, bottom, left isometric view of the head immobilization aid of FIG. 1 as applied to a patient's head with the straps and flaps closed.

FIG. 7 is a rear, bottom, right isometric view of the head immobilization aid of FIG. 1 as applied to a patient's head according to another implementation.

FIG. 8 is a top left view of the head immobilization aid of FIG. 1 as applied to a patient's head with the straps closed and a medical device secured to the straps.

FIG. 9 is a front, bottom, right view of the head immobilization aid of FIG. 1 as applied to a patient's head with the straps and flaps closed and a medical device secured to the straps and flaps.

### DETAILED DESCRIPTION

Head Immobilization Aid

Head immobilization aids primarily for patients who could benefit from head immobilization are disclosed herein. The head immobilization aids may be generally understood as having immobilization members, a rear panel, at least one strap, and at least one flap. The immobilization members maintain a patient's head in a desired position, such as midline. The one or more straps assist in securing the head immobilization aid to a patient's head. The one or more flaps assist in securing the immobilization members in a desired position. In some implementations, access to the scalp, such as for placement, removal, or monitoring of intravenous (IV) catheters administered on the scalp and for regulating temperature, is maintained while the head positioning aid is in use. In some implementations, a medical device may be secured to the head immobilization aid.

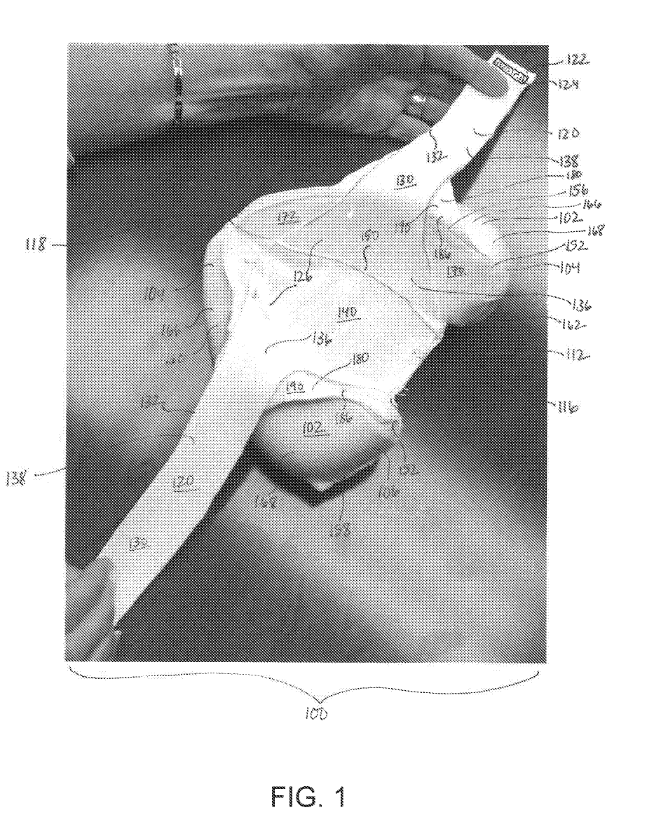

FIGS. 1-7 depict one embodiment of a head immobilization aid 100. The head immobilization aid 100 comprises immobilization members 102 and at least one strap 120, all secured to a rear panel 140, and at least one lateral flap 180 secured to an immobilization member 102. While two immobilization members 102 are shown in the figures as separate members spaced laterally apart from each other, a single U-shaped or V-shaped immobilization member could also be used so long as two lateral support members are provided as part of the single immobilization member along lateral sides of the head to prevent side-to-side movement or rolling thereof.

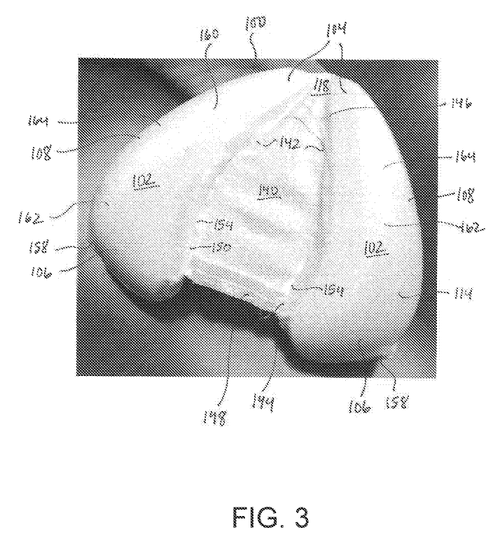

The head immobilization aid 100 of the exemplary embodiment depicted in FIGS. 1-7 has an apex 118, a front side 112, and a back side 114. In one embodiment, and as depicted in FIG. 3, the back side 114 includes a rear panel 140. The rear panel has a rear upper portion 146, a rear lower portion 144, and rear lateral edges 142. The rear lower portion 144 may include one or more hems 148.

A hem 148 may extend partially or completely across the rear lower portion 144. When the head immobilization aid 100 is worn by a patient, the rear lower portion 144 may lie near or on the base of the patient's skull and above the nape of the neck.

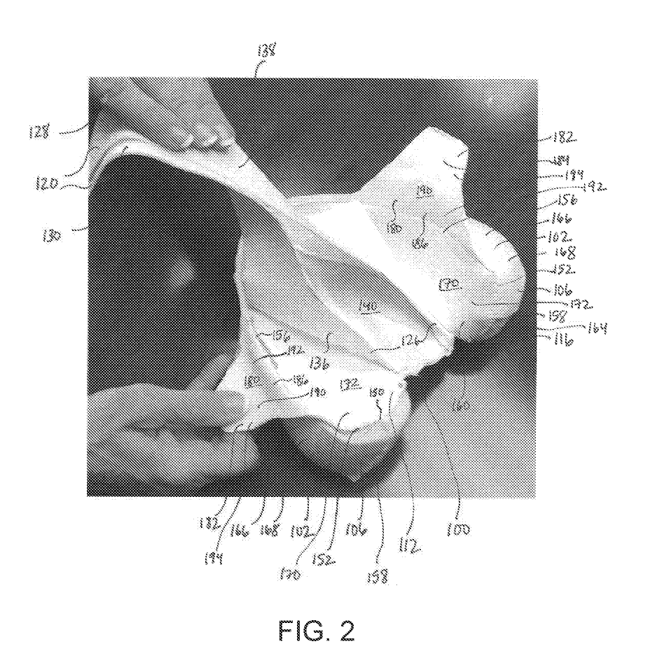

In the exemplary embodiment depicted in FIGS. 1 and 2, the front side 112 of the head immobilization aid 100 has a lower portion 116, at least one strap 120, and at least one lateral flap 180. When the head immobilization aid 100 is worn by a patient, the lower portion 116 may lie near or on the patient's ears.

In one embodiment, at least one strap 120 is attached at a fixed end 126 to the rear panel 140 on the front side 122 of the head immobilization aid 100. In the exemplary embodiment of FIGS. 1 and 2, one strap 120 extends from each of the lateral sides of the rear panel 140 on the front side 122 of the head immobilization aid 100. Each strap 120 is substantially Y-shaped with the fixed end 126 being the top or wider portion 136 and the free end 122 being the narrower or stem portion 138. The wider portion 136 may be a full panel or web of fabric from edge to edge (rather than having a void triangular center between two arms as in a true “Y-shape”) that extends from at or near the apex 118 to at or near the lower portion 116 and may have a curved shape that generally follows the contour of the side of a patient's head. The shape of the strap 120 may taper quickly from the wider portion 136 to the stem portion 138. The free end 122 may be substantially rectangular in shape with its length greater than its width. In one embodiment, each strap 120 is 2 to 20 inches in length (e.g., 3 to 18 inches in length, 4 to 16 inches in length, 6 to 14 inches in length, or 8 to 12 inches in length, e.g., 2 to 4 inches in length, 4 to 6 inches in length, 6 to 8 inches in length, 8 to 10 inches in length, 10 to 12 inches in length, 12 to 14 inches in length, 14 to 16 inches in length, 16 to 18 inches in length, or 18 to 20 inches in length). In one embodiment, the narrower portion 138 of each strap 120 is 0.5 inches to 4 inches in width (e.g., 0.8 inches to 3.5 inches in width, 1.0 to 3.0 inches in width, or 1.5 to 2.5 inches in width, e.g., 0.5 inches to 1.0 inch in width, 1.0 inch to 1.5 inches in width, 1.5 to 2.0 inches in width, 2.5 to 3.0 inches in width, 3.0 to 3.5 inches in width, or 3.5 to 4.0 inches in width).

The immobilization members 102 may be comprised of a filler encased within respective pockets. The filler may be, for example, solid foam, memory foam, stuffing, batting, down, synthetic down-like material, gel, or a combination thereof. The filler may be resilient such that it returns to its original shape, or close to its original shape, after being compressed. The filler may be of sufficient quantity, compactness, or firmness that it resists complete compression by the weight of a patient's head. The filler may be partially compressible, but has enough compression resistance that it helps each immobilization member 102 minimize movement of a patient's head and deflect a patient's head moving head back to a desired position. The compression resistance may be matched to the size and weight of a patient's head, or to the force exerted by a patient's moving head. For example, the filler may compress to about fifty percent or less of its uncompressed thickness when weighted under a patient's head.

In some embodiments, the firmness of the immobilization member, as measured by indentation force deflection (IFD) required to make a dent 1 inch into a foam sample of 15 inches×15 inches×4 inches by an 8 inch diameter disc, is from 5 pounds to 25 pounds (e.g., 5 pounds to 10 pounds, 10 pounds to 12 pounds, 12 pounds to 14 pounds, 14 pounds to 16 pounds, 16 pounds to 18 pounds, 18 pounds to 20 pounds, or 20 pounds to 25 pounds).

In some embodiments, the density of the immobilization member is from 0.1 to 20 pounds per cubic foot (e.g., from 0.5 to 10 pounds per cubic foot or from 1 to 5 pounds per cubic foot, or from 0.1 to 0.5 pounds per cubic foot, from 0.5 to 1.0 pounds per cubic foot, from 1.0 to 2.0 pounds per cubic foot, from 2.0 to 3.0 pounds per cubic foot, from 3.0 to 4.0 pounds per cubic foot, from 4.0 to 5.0 pounds per cubic foot, from 5.0 to 10 pounds per cubic foot, or from 10 to 20 pounds per cubic foot).

In some embodiments, the immobilization members 102 are inflatable, such as with air. Each immobilization member 102 may include a bladder connected to a valve. The valve or a tube connected to the valve may be accessible from the outer surface of the immobilization member 102, which allows the bladder to be inflated. The bladder may be inflated manually, such as by blowing into the valve or tube, or may be inflated mechanically, such as with a pump supplying compressed air.

Each bladder may be filled such that it has sufficient volume or firmness to resist complete compression by the weight of a patient's head. The bladders may be filled such that they help the immobilization members 102 minimize movement of a patient's head, maintain a patient's head in a desired position, and deflect a patient's moving head back to a desired position. The fill amount may be matched to the size and weight of a patient's head, or to the force exerted by a patient's moving head. In some implementations, one bladder may be deflated or filled to a volume that permits the patient's head to partially or completely compress the corresponding immobilization member 102 and the other bladder may be inflated to a volume that directs the patient's head towards the first deflated immobilization member 102. The bladders may be inflated or deflated in an opposing coordinated manner to move the head of a patient from side to side at any desired time interval.

Each immobilization member 102 may have an overall curved or kidney-bean shape with a tapered end. The immobilization members 102 may each have the same shape or have different shapes, and may each have the same size or be of different sizes. The size of the immobilization members 102 may be commensurate with the size of the patient's head to which the head immobilization aid is secured. Each immobilization member 102 has an upper end 104 positioned at or near the apex 118 of the head immobilization aid 100, and has a lower end 106 positioned at or near the lower portion 116 of the head immobilization aid 100. The tapered end of the immobilization member 102 may be at the upper end 104.

Each immobilization member 102 may be the same length as, shorter than, or longer than, the length of the head immobilization aid 100 at the location to which that immobilization member 102 is secured. Thus, the upper end 104 may extend beyond the apex 118 of the head immobilization aid 100, extend to the apex 118, or terminate below the apex 118. The lower end 106 may extend beyond the lower portion 116 of the head immobilization aid 100, extend to the lower portion 116, or terminate above the lower portion 116.

Each immobilization member 102 is elongated such that its length is greater than either its width or depth (thickness). The longitudinal elongation may help an immobilization member 102 maintain a patient's head in a desired position and deflect a patient's moving head back to a desired position.

Each immobilization member 102 extends laterally outward from the outer surface of the rear panel 140. Each immobilization member 102 may have an extension distance (width) greater at its lower end 106 than at its upper end 104. For example, an immobilization member 102 may have nominal width at the upper end 104 and a functional width at its lower end 106. The width at the lower end 106 may be enough to help the immobilization member 102 maintain a patient's head in a desired position and deflect a patient's moving head back to a desired position. In one embodiment, the lower end 106 may be 1.5 inches to 6 inches wide (e.g., 2 inches to 5 inches wide or 3 inches to 4 inches wide, e.g., 1.5 inches to 2 inches wide, 2 inches to 3 inches wide, 3 inches to 4 inches wide, 4 inches to 5 inches wide, or 5 inches to 6 inches wide).

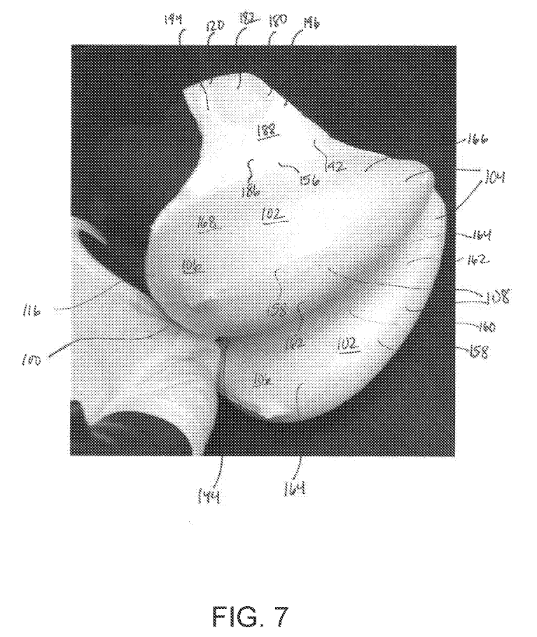

An immobilization member 102 may have the same thickness or depth along the entire length of the immobilization member 102 or, as shown in FIG. 7, the thickness may vary along the length of the immobilization member 102. An immobilization member 102 may be thicker at its lower end 106 than at its upper end 104. The lower end 106 may be 1.25 to 6 times as thick as the upper end 104. The thickness at the lower end 106 may be enough to help the immobilization member 102 maintain a patient's head in a desired position and deflect a patient's moving head back to a desired position.

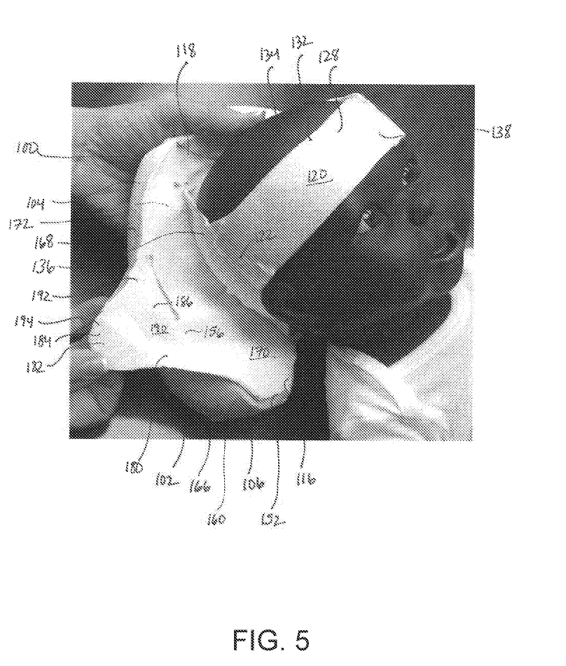

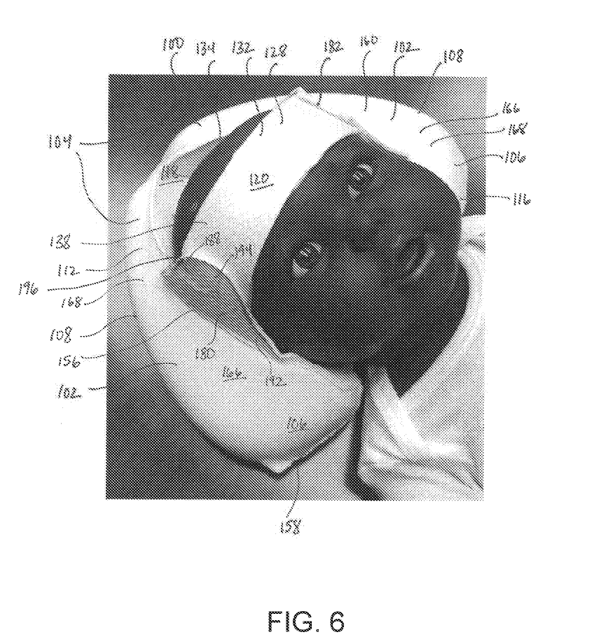

Each immobilization member 102 may have a rear face 162, a front face 166, and an inner face 170. With reference to FIG. 3, the rear face 162 may be adjacent a lateral edge 142 of the rear panel 140. The rear face 162 may be substantially planar such that when it is placed on a flat surface, a majority of the rear face 162 contacts the surface. When the head immobilization aid 100 is used with two immobilization members 102 arranged adjacent to each other (see FIG. 7), a majority of the rear face 162 of each immobilization member 102 may be in contact with the other rear face 162. The front face 166 may be substantially planar or curved. With reference to FIG. 6, the front face 166 is adjacent a top surface 188 of a lateral flap 180 when the head immobilization aid 100 is in use. The inner face 170 may be substantially flat or smooth but the overall shape is curved (e.g., like a parabolic cylinder section) to generally follow the curvature of the sides of a patient's skull. The inner face 170 may be adjacent a top surface 128 of a wider portion 136 of a strap 120 when the head immobilization aid 100 is in use and the straps 120 and lateral flaps 180 are in the closed position (see FIG. 5). Each immobilization member 102 is secured to the rear panel 140 of the head immobilization aid 100.

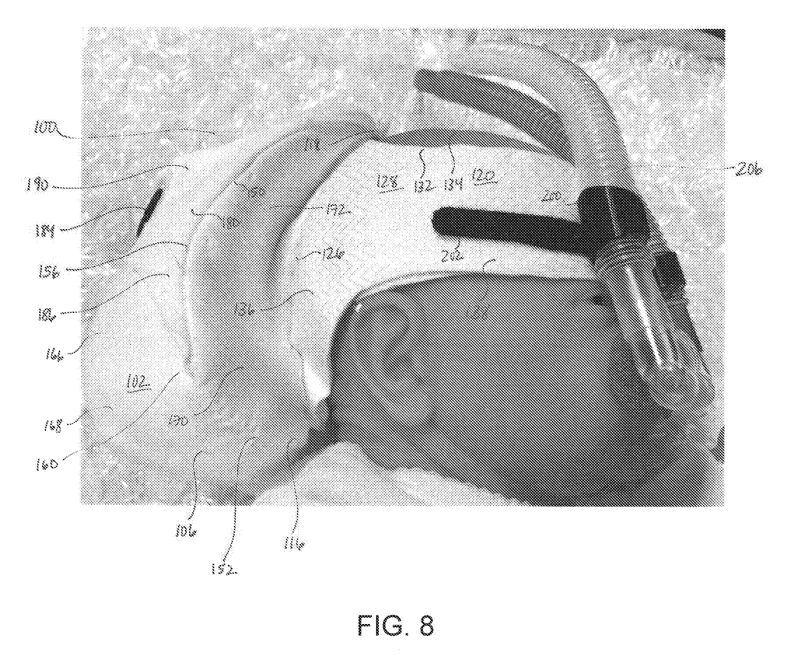

In one embodiment, at least one lateral flap 180 is attached at a fixed end 186 to an immobilization member 102 at or near the intersection of the inner face 170 and front face 166, such as at a front juncture 156. In some embodiments, and with reference to FIG. 8, the lateral flap 180 and inner face 170 of the immobilization member 102 are constructed of a single piece of fabric and the front juncture 156 perforates the lateral flap 180 and inner face 170 portions. In the exemplary embodiment of FIGS. 2 and 5, one lateral flap 180 extends from each of two immobilization members 102. Each lateral flap 180 may be substantially bell-shaped or wing-shaped with the fixed end 186 being the wider or base portion 192 and the free end 182 being the tip or narrower portion 194. In one embodiment, the base portion 192 extends from at or near the upper end 104 of the immobilization member 102 to at or near the lower end 106. In another embodiment, the base portion 192 extends for approximately the middle third of the length of the immobilization member 102. The narrower portion 194 may have a tab-shape at the free end 182, which may help medical personnel easily grip the free end 182.

Each of the rear panel 140, straps 120, and lateral flaps 180 may be constructed of any of one or more soft fabric materials known in the art. The filler or bladder of each immobilization member 102 may be partially or completely surrounded by a fabric pocket, cover, or casing 160 constructed of one or more of the fabric materials. The fabric material may be any natural or synthetic fabric such as cotton, elastane or spandex, microfiber, polyester, rayon, silk, viscose, or wool, or any combination thereof. The fabric material may be a composite of open-celled, elastomeric, non-latex foam and engineered fabrics. For example, the composite may be FABRIFOAM® (Fabrifoam, Exton, Pa.). The fabric may be woven, unwoven, or knit. A knit may be a smooth or ribbed knit. The material may be flexible, stretchable, migration resistant, hook receptive (i.e., micro-hooks from hook-and-loop fastener materials will attach to the material), wicking, breathable, cooling, fire retardant, machine washable, or any combination thereof.

In the construction and use of the rear panel 140, straps 120, or lateral flaps 180, a material that is stretchable may help secure the head immobilization aid 100 to a patient's head. A material that is stretchable may also provide versatile adjustability in both circumference and height of the head immobilization aid 100. A stretchable material may evenly apply an elastic-like grip around the circumference of the head immobilization aid 100 for a comfortable fit that is also resistant to unintentional movement, such as rotating around a patient's head or slipping up or down on a patient's head.

Stretchable materials may include stretchable fabrics such as, for example, elastane or spandex, nylon, and ribbed knits. Fabric weaves with a combination of stretchable fabric threads and other fabrics such as those identified above may result in a composite fabric with greater stretch than the base fabric alone. The stretch of a fabric may be limited by structures such as seams. The stretch of a fabric may also be limited by increasing the number of layers of fabric or overlaying a stretchable fabric and a non-stretchable fabric. Alternately, the fabric may be reinforced with elastic strips or bands that grip the patient's head.

In the construction and use of the rear panel 140, straps 120, or lateral flaps 180, a migration-resistant fabric may help the head immobilization aid 100 remain in place on a patient's head and may help reduce or prevent the unintentional movement of the head immobilization aid 100, such as rotating around a patient's head or slipping up or down on a patient's head. Migration-resistant fabrics cling to or grip the surface with which they are in contact. Migration-resistant fabrics may include, for example, spandex and FABRIFOAM®.

In the construction and use of the rear panel 140, straps 120, or lateral flaps 180, a hook-receptive fabric may reduce the number of fasteners needed to secure the head immobilization aid 100 to a patient's head. Hooks, such as Velcro® hooks, can directly engage hook-receptive fabrics. Hook-receptive fabrics may include, for example, fleece, flannel, terrycloth, and FABRIFOAM®.

In the construction and use of the rear panel 140, straps 120, or lateral flaps 180, a non-insulating fabric may help prevent a patient's body temperature from rising or reduce a patient's body temperature as compared to an insulating fabric. Non-insulating fabrics include fabrics that are wicking, breathable, and/or cooling.

A wicking fabric draws moisture away from skin and may also transfer it to a next, more outer, layer. Drawing moisture, usually perspiration, away from the skin helps regulate body temperature. For example, drawing moisture away from the skin helps a person feel or stay warmer in cool or cold environments and helps a person feel or stay cooler in warm or hot environments. A wicking fabric may help a patient regulate body temperature. Wicking fabrics may include, for example, cotton, microfiber, polyester, silk, and wool. Wicking fabrics may also include performance-engineered synthetic fabrics such as FABRIFOAM®, CAPILENE® (Patagonia, Ventura, Calif.), FLASHDRY™ (The North Face, San Leandro, Calif.) and DRICLIME® (Marmot, Rohnert Park, Calif.).

A breathable fabric allows air to reach the skin and allows water vapor to escape from the fabric. Allowing air to reach the skin and allowing water vapor, usually from perspiration, to escape from the fabric helps to reduce body temperature and/or prevent body temperature from rising. A breathable fabric may help a patient stay cooler. Breathable fabrics may include, for example, cotton, linen, and silk. Breathable fabrics may also include performance-engineered synthetic fabrics such as FABRIFOAM®, Gore-Tex® (breathable and waterproof; W. L. Gore & Associates, Elkton, Md.), OMNITECH® (breathable and waterproof; Colombia Sportswear Co., Portland, Oreg.) and POLARTEC® (breathable and insulating; Marmot, Rohnert Park, Calif.).

A cooling fabric allows heat to pass away from the skin through the fabric and does not reflect heat back to the skin. Allowing heat to pass through the fabric helps to reduce body temperature and/or prevent body temperature from rising. A cooling fabric may help a patient stay cooler. Cooling fabrics may include, for example, cotton, linen, and rayon.

Each of the rear panel 140, straps 120, lateral flaps 180, and casing 160 may be constructed of one or more layers of soft materials, such as one layer or two layers. Each layer may be constructed of one or more pieces joined at a seam or juncture 150. A juncture 150 may be formed by any known means including, but not limited to, stitching, glue, tape, bonding, or any combination thereof. In the exemplary embodiment depicted in FIGS. 1 and 3, each strap 120 is constructed of one piece of FABRIFOAM® and the rear panel 140 is constructed of one piece of cotton.

In the exemplary embodiment depicted in FIGS. 2, 3, and 7, each casing 160 is constructed of three pieces of fabric. A rear casing 164 is adjacent to or covers the rear face 162, a front casing 168 is adjacent to or covers the front face 166, and an inner casing 172 is adjacent to or covers the inner face 170. The rear casing 164 and front casing 168 may be constructed of cotton and the inner casing 172 may be constructed of FABRIFOAM®. Each piece of the casing 160 is joined to at least one other piece of casing 160, or to another portion of the head immobilization aid 100, at a juncture 150. A juncture 150 may extend partially or completely along a given dimension of an immobilization member 102 and may traverse more than one dimension. For example, and with reference to FIGS. 1 and 2, a lateral juncture 158 may extend from the apex 118, along the outer side 108 of an immobilization member 102 for the entire length of the immobilization member 102, and then curve under the lower end 106 of the immobilization member 102.

The rear casing 164 is joined to a lateral edge 142 of the rear panel 140 at a rear juncture 154. The rear casing 164 is also joined to the front casing 168 at a lateral juncture 158 and to the inner casing 172 at a lower juncture 152. The front casing 168 is joined to the inner casing 172 at the lower juncture 152 and is joined to a lateral flap 180 at a front juncture 156. The inner casing 172 is joined to a lateral flap 180 at the front juncture 156 and joined to the rear panel 140 at a rear juncture 154.

Securing the rear casing 164 and inner casing 172 to the rear panel 140 at the rear juncture 154 helps to secure the immobilization member 102 to a lateral edge 142 of the rear panel 140. In the construction and use of the head immobilization aid 100, securing the immobilization members 102 to the rear panel 140 along a single juncture 150, such as the rear juncture 154, helps create a hinge-like arrangement of the immobilization member 102 relative to the rear panel 140. The immobilization member 102 can pivot nearly 360° around the rear juncture 154. The wide range of movement helps permit highly versatile positioning and adjustability of the immobilization member 102.

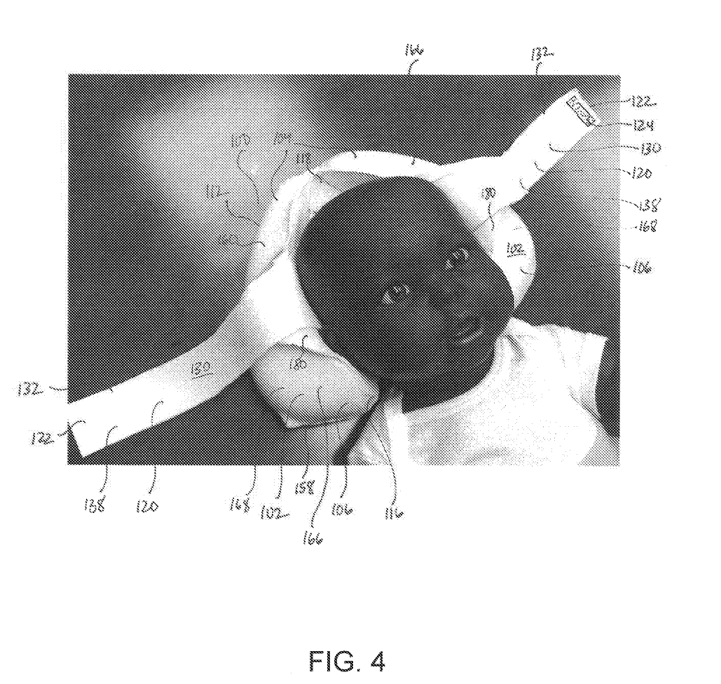

In the exemplary embodiment depicted in FIGS. 1-6, an immobilization member 102 is positioned on each of the left and right sides of the head immobilization aid 100. The immobilization members 102 meet or nearly meet at or near the apex 118. With reference to FIGS. 4 and 5, the immobilization members 102 are positioned over the ears of a patient when the head immobilization aid 100 is in use.

The immobilization members 102 minimize movement of a patient's head, maintain a patient's head in a desired position, and deflect a patient's moving head back to a desired position. The immobilization members 102 may be positioned laterally when a patient's head is in a supine midline position, which diminishes pressure to the back and sides of the head.

The straps 120 may be opened to apply or remove the head immobilization aid 100. The straps 120 may be closed to help secure the head immobilization aid 100 to the head of a patient. The straps 120 may be closed to help provide a surface to which medical devices can be attached. When the straps 120 are in the open position (see FIGS. 1 and 4), the straps 120 may be folded back such that they lay on top of a portion of the lateral flaps 180 or immobilization members 102. The straps 120 may be wrapped underneath the immobilization members 102.

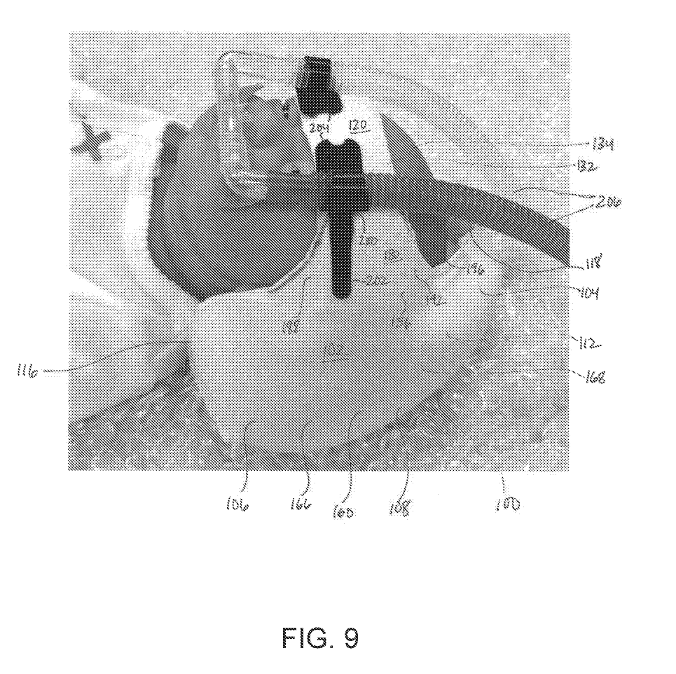

When the straps 120 are in the closed position (see FIGS. 5 and 6), the straps 120 lay substantially flat on the patient's skull. In some embodiments, the combined length of the straps 120 is at least long enough to traverse the distance between rear junctures 154 across the forehead of a patient when the head immobilization aid 100 is in use and the straps 120 are in the closed position. In the exemplary embodiment of FIG. 5, each strap 120 is long enough to traverse the patient's forehead and meet the wider portion 136 of the opposing strap 120 when the head immobilization aid 100 is in use and the straps 120 are in the closed positon. The width of each strap 120 may be wide enough to receive a medical device, such as a CPAP tube 206, or to receive an attachment device 200 that helps secure a medical device to the strap 120 (see FIG. 9). The width of each strap 120 is not so wide as to cover the crown of a patient's head when the head immobilization aid 100 is in use and the straps 120 are in the closed positon.

The straps 120 may be releasably secured to each other or to a lateral flap 180 at one or more strap attachments 124. The strap attachments 124 may include, for example, buttons, snaps, hook-and-loop fasteners, or hook-and-eye fasteners. In the exemplary embodiment of FIG. 1, the strap attachment 124 is constructed of hook fasteners and is positioned on a bottom surface 130 at or near the free end 122 of a strap 120. In some embodiments, an opposing strap includes a second strap attachment 124, such as one formed by an area of loop fasteners positioned on a top surface 128 of the opposing strap 120.

With reference to the exemplary embodiment depicted in FIGS. 1 and 5, the top surface 128 of the straps 120 is constructed of a hook-receptive material. A hook strap attachment 124 on the bottom surface 130 of one strap 120 can be secured to the hook-receptive top surface 128 of the opposing strap 120 by pressing the hook strap attachment 124 against the opposing strap 120. In some embodiments, the top surface of the lateral flap 180 is constructed of a hook-receptive material. In an alternative embodiment, a hook strap attachment 124 on a strap 120 can also engage a hook-receptive surface on the lateral flap 180.

Securing the straps 120 helps to secure the head immobilization aid 100 to a patient's head. Securing the straps 120 may help the head immobilization aid 100 remain in place on a patient's head and may help reduce or prevent the unintentional movement of the head immobilization aid 100, such as rotation around or slippage up or down on a patient's head. Securing the straps 120 may help provide a surface on which one or more medical devices or attachment devices 200 for medical devices can be placed. In the construction and use of the straps 120, their shape, material, and position help protect the skin of a patient's face from abrasion or other damage caused by a medical device, such as a CPAP tube 206, or an attachment device 200, touching or laying on the face.

With reference to FIGS. 5 and 6, when the straps 120 are in the closed position, the upper edges 132 of the straps 120 define a portion of an upper opening 134 in the head immobilization aid 100. The rear panel 140 and apex 118 define another portion of the upper opening 134. In some embodiments, when the lateral flaps 180 are in the closed position, the upper edge 196 of the lateral flaps 180 defines a portion of the upper opening 134. The upper opening 134 may be substantially circular or oval in shape. When the head immobilization aid 100 is in use, the upper opening 134 is positioned over the top of the patient's head above the forehead. The upper opening 134 provides access to the scalp, which allows for placement, removal, and monitoring of scalp IVs, electrodes (e.g., for electroencephalogram (EEG) testing) or other medical instrumentation. The upper opening 134 also permits temperature monitoring, regulation, and stabilization.

The lateral flaps 180 may be in either a fastened or released position when applying or removing the head immobilization aid 100. A flap 180 may be closed to help secure the adjacent immobilization member 102 in a desired position relative to the rear panel 140. The flaps 180 may also be closed to help secure the head immobilization aid 100 to the head of a patient.

When the lateral flaps 180 are in a released position (see FIGS. 1, 4, and 8), the flaps 180 may be folded back such that they lay on top of a portion of the immobilization members 102. When the lateral flaps 180 are in a fastened position (see FIG. 6), the bottom surface 190 of the flaps 180 lays substantially flat on the top surface 128 of the straps 120. The width of a lateral flap 180 at its widest point, such as from the front juncture 156 to the free end 182, is long enough to secure the flap 180 to a strap 120. In the exemplary embodiment of FIG. 6, when the head immobilization aid 100 is in use and the straps 120 and lateral flaps 180 are in the fastened position, the flap 180 is long enough to extend beyond the point of engagement of the strap attachment 124 of the distally attached strap 120 to the proximally attached strap 120. With further reference to FIG. 6, the length of each lateral flap 180 is enough to cover the patient's ear when the head immobilization aid 100 is in use and the straps 120 and flaps 180 are in the fastened positon.

In the design, construction, and use of the head immobilization aid 100, the attachment of a lateral flap 180 to an immobilization member 102 at a single juncture 150, such as the front juncture 156, permits the flap 180 to direct the adjacent immobilization member 102 into any desired position. For example, lifting the free end 182 of a lateral flap 180 towards the front side 112 of the head immobilization aid 100 draws the adjacent immobilization member 102 towards the patient's face. Pulling the free end 182 of the flap 180 towards the center of the patient's face draws the inner face 170 of the immobilization member 102 towards the patient's ears. Lifting up and in on the free end 182 draws the inner face 170 further around or tighter against the side of the patient's face. Releasing the free end 182 or drawing it towards the back side of the head immobilization aid 100 permits the immobilization member 102 to fall further back along the side of the head or outward away from the side of the head. The lateral flap 180 is operably connected to the rear panel 140 through the inner face 170 of the immobilization member 102. Pulling outward or upward on the lateral flap 180 stretches the rear panel 140 and releasing the flap 180 loosens the rear panel 140.

Each lateral flap 180 may be releasably secured to a strap 120 by one or more flap attachments 184. The flap attachments 184 may include, for example, buttons, snaps, hook-and-loop fasteners, or hook-and-eye fasteners. In the exemplary embodiment of FIG. 2, the flap attachment 184 is constructed of hook fasteners and is positioned on a bottom surface 190 at or near the free end 182 of a flap 180. In some embodiments, a second flap attachment 184, such as one constructed of loop fasteners, may be positioned on a top surface 128 of a strap 120.

With reference to the exemplary embodiment depicted in FIGS. 2 and 6, the top surface 128 of the straps 120 may be constructed of a hook-receptive material. A hook flap attachment 184 on the bottom surface 190 of one flap 180 can be secured to the hook-receptive top surface 128 of a strap 120 by pressing the hook flap attachment 184 against the strap 120.

Securing a lateral flap 180, such as to a strap 120, helps secure the adjacent immobilization member 102 in a desired position relative to the rear panel 140. When the lateral flap 180 is unsecured, the adjacent immobilization member 102 is able to pivot about the rear juncture 154. When the lateral flap 180 is secured, the rotational movement of the immobilization member 102 may be restricted. The relative positions of the rear panel 140, rear juncture 154, inner face 170, front juncture 156, and lateral flap 180 are maintained by securing the flap 180. Securing the flap 180 also helps limit stretching of the rear panel 140, which helps limit relative movement between the immobilization members 102. In some embodiments, and with reference to FIGS. 7 and 8, when a patient is in a side lying or partial side lying position, the immobilization members 102 may be positioned behind the head with the rear faces 162 pushed together such that the rear panel 140 is slack. In these embodiments, securing the lateral flaps 180 to the straps 120 helps limit movement of the immobilization members 102 relative to each other.

At any time after the lateral flaps 180 have been secured, the flaps 180 may be repositioned by releasing the flap attachment 184, directing the flap 180, such as by pulling on the free end 182, to a new position, and re-securing the flap 180 at a different location on the strap 120. Repositioning the flap 180 repositions the adjacent immobilization member 102 with respect to the rear panel 140, straps 120, and patient's head. Positioning an immobilization member can help positon a patient's head in a desired position, such as midline, and repositioning an immobilization member can help re-positon a patient's head in a different desired position.

Securing the lateral flaps 180 may also help to secure the head immobilization aid 100 to a patient's head. Securing the flaps 180 may help the head immobilization aid 100 remain in place on a patient's head and may help reduce or prevent the unintentional movement of the head immobilization aid 100, such as rotation around or slippage up or down on a patient's head.

### Methods of Use of the Head Immobilization Aid

By way of example, but not limitation, the head immobilization aid 100 of FIGS. 1-6 may be used to support a patient in a desired position, such as the midline supine position, according to the following procedure. A patient, such as a baby, is placed on its back and its head is positioned on the rear panel 140 of a head immobilization aid 100 with the straps 120 in the released position, or the rear panel 140 of the head immobilization aid 100 is guided underneath the baby's head. Placing the patient on the head immobilization aid 100 or sliding the head immobilization aid 100 down the back of the baby's head minimizes disturbance to and stress on the baby. A similar procedure may be followed for placing the head immobilization aid 100 on an older child or adult. Alternatively, an adult patient may self-position on the head immobilization aid 100.

The head immobilization aid 100 is positioned with the back side 114 of the headwear member 110 facing the surface on which the patient is placed. The apex 118 of the headwear member 110 is positioned behind the crown of the head. The rear lower portion 144 of the rear panel 140 is positioned behind the back of the neck.

The free end 122 of a strap 120 is drawn across the patient's forehead towards the opposing immobilization member 102 and is laid substantially flat against the patient's forehead. The bottom surface 130 of the strap 120 contacts the patient's skin without irritation or damage. When a second strap 120 is present, the free end 122 of the second strap 120 is drawn towards the opposing immobilization member 102 and is laid substantially flat against the patient's forehead or on top of the top surface 128 of the previously positioned strap 120. The second strap 120 is secured to the first strap 120 by pressing a strap attachment 124 positioned on the bottom surface 130 of the second strap 120 against the hook-receiving top surface 128 of the first strap 120 to engage the two surfaces 128, 130.

When the straps 120 are engaged, the upper edges 132 define a portion of an upper opening 134. The upper opening 134 permits access to the scalp for placement, removal, or monitoring of scalp IVs, electrode, or other medical devices and for regulating temperature. The side of patient's face and the ears remain exposed when the straps 120 are engaged and the flaps 180 are not engaged.

The free end 182 of a lateral flap 180 is drawn up and in toward the center of the patient's forehead. The immobilization member 102 to which the flap 180 is attached is also drawn in toward the side of the head. When the free end 182 is pulled minimally, the immobilization member 102 remains pivoted outward from the side of the head or the immobilization member 102 remains further back along the side of the head, such as behind the ear. When the free end 182 is pulled taught, the attached immobilization member 102 is rotated in toward the side of the head such that the inner face 170 covers the ear.

The bottom surface 190 of each lateral flap 180 that is drawn in may be laid substantially flat against the top surface 128 of a strap 120. The lateral flap 180 is secured to the strap 120 by pressing a flap attachment 184 positioned on the bottom surface 190 of the flap 180 against the hook-receiving top surface 128 of the strap 120 to engage the two surfaces 128, 190.

A lateral flap 180 is later repositioned by releasing the flap attachment 184, pulling on or releasing the free end of the flap 180, and re-securing the flap 180 at a different location on the strap 120. Repositioning the flap 180 repositions the adjacent immobilization member 102 with respect to the rear panel 140, straps 120, and patient's head.

The immobilization members 102 are positioned on either side of the patient's head, centered over the ears and extending in front of or behind the ears. The immobilization members 102 maintain a patient's head in a desired position and deflect a patient's moving head back to a desired position. The inner face 170 of the immobilization members 102 follows the curvature of the patient's head such that the lower portions 116 may fall behind or in front of the tops of the shoulders. This arrangement of the lower portions 116 helps provide additional lateral support to the immobilization members 102 for maintaining the head in a desired position. When the patient's head rests on or rolls onto an immobilization member 102, the immobilization member 102 deflects the patient's head back to the desired position. The shape, thickness, and/or firmness of the immobilization members 102 may help to deflect a patient's head back to the desired position. Maintaining a patient's head in midline helps to promote optimal cerebral blood flow and uniform distribution of respiratory support to the lungs. Maintaining a patient's head in midline or other desired positions permits the effective and unobstructed delivery of other medical interventions, such as CPAP, and permits recovery from a head or neck injury.

The head immobilization aid 100 of FIGS. 1-3 and 8 may be used to support a patient in a desired position, such as a midline position, while the patient is lying on its side according to the following procedure. The head immobilization aid 100 is positioned and secured to the patient's head as described above for FIGS. 1-6 except that when the patient is lying on its side, the immobilization members 102 may be drawn together behind the patient's head, and the rear faces 162 of the immobilization members 102 may be in contact with each other. The rear panel 140 is slack between the immobilization members 102. The lateral flap 180 on the side on which the patient is lying may be unsecured or may be secured to a strap 120 before the patient is placed on top of the head immobilization aid 100. The exposed lateral flap 180 may be secured as described above. The secured lateral flap 180 or flaps 180 help maintain the immobilization members 102 in relative position to one another when the rear panel 140 is slack. The immobilization members 102 support and maintain a patient's head in a midline position while the patient is lying on its side.

The head immobilization aid 100 of FIGS. 1-3 and 7 may be used to support a patient in a desired position, such as a partial side lying positon, according to the following procedure.

The head immobilization aid 100 is positioned and secured to the patient's head as described above for FIGS. 1-3 and 8 except that the patient is lying on its back with its head turned to the side. The immobilization members 102 support and maintain a patient's head in a partial side lying position.

As another example, the head positioning aid 100 of FIGS. 1-3 may be used with an attachment device 200 as shown in FIGS. 8 and 9 to support a patient's head in a desired position, as well as to secure straps, wires, lines, or tubes of or connected to medical devices and to guide them away from a patient's face, according to the following procedure.

The head immobilization aid 100 may be applied to a patient's head according to any of the methods described above. An attachment device 200 shaped like a zip tie and having a hook fastener surface is positioned under a previously placed strap, wire, line, or tube such that the hook fastener surface faces the head immobilization aid 100. The tail portion 202 of the attachment device 200 is drawn around, for example, a CPAP tube 206, passed through an aperture in the head portion 204, drawn away from the head portion 204, and pulled to tighten the attachment device 200 around the tube 206. The head portion 204 and exposed portion of the tail portion 202 are pressed against the hook-receptive top surface 128 of the closed strap 120 or lateral flap 180 at any desired location. Other attachment and guide systems or structures for positioning and routing of medical devices may be attached to the head immobilization aid 100, e.g., on the straps 120, by similarly using a hook fastening surface on the attachment or guide system.

Thus, the invention provides a head immobilization device for use according to any of the methods described supra.

### Head Immobilization Aid Kits

By way of example, but not limitation, the head immobilization aid 100 of FIGS. 1-3 may be provided as part of a kit. A kit can include a head immobilization aid 100 and one or more of an attachment device 200, launderable bag, and instructions for using the head immobilization aid 100 or launderable bag. The head immobilization aid 100 can be transported, stored, or washed in the launderable bag.

The article “a” or “an” preceding a term, as used herein, refers to one or more of that term. As such, the terms “a” or “an”, “one or more”, and “at least one” should be considered interchangeable herein.

All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the present invention, and do not create limitations, particularly as to the position, orientation, or use of the invention. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. The exemplary drawings are for purposes of illustration only and the dimensions, positions, order and relative sizes reflected in the drawings attached hereto may vary.

The above specification, examples and data provide a complete description of the structure and use of exemplary embodiments of the invention as defined in the claims. Although various embodiments of the claimed invention have been described above with a certain degree of particularity, or with reference to one or more individual embodiments, those skilled in the art could make numerous alterations to the disclosed embodiments without departing from the spirit or scope of the claimed invention. Other embodiments are therefore contemplated. It is intended that all matter contained in the above description and shown in the accompanying drawings shall be interpreted as illustrative only of particular embodiments and not limiting. Changes in detail or structure may be made without departing from the basic elements of the invention as defined in the following claims.

What is claimed is:

1. A head immobilization aid comprising
a rear panel having lateral edges;
first and second straps attached to opposing lateral edges of the rear panel, the straps configured to secure the head immobilization aid to a patient's head by traversing the patient's forehead from opposing sides and being secured together;
two immobilization members spaced laterally apart from each other on opposing lateral edges of the rear panel and attached to said opposing edges; and
a flap attached to each of the two immobilization members, wherein
each immobilization member has a back face adjacent to a lateral edge of the rear panel and an inner face adjacent to and attached to a flap;
the flaps are configured to secure the immobilization members in a desired position relative to the rear panel when the flaps are secured to the straps; and
the flaps may be repositioned with respect to the straps to allow hinged movement of the immobilization members relative to the lateral edges of the rear panel to reposition the immobilization members relative to the rear panel, the straps, and the patient's head.

2. The head immobilization aid of claim 1, wherein each immobilization member is secured to the rear panel at a single juncture.

3. The head immobilization aid of claim 1 or 2, wherein each flap is secured to the adjacent immobilization member at a single juncture distal from the juncture between the same immobilization member and the rear panel.

4. The head immobilization aid of any one of claims 1-3, wherein the back face of each immobilization member is substantially flat.

5. The head immobilization aid of any one of claims 1-4, wherein the inner face of each immobilization member is contoured to conform to a side of a human head.

6. The head immobilization aid of any one of claims 1-5, wherein the flap has a fixed end secured to the adjacent immobilization member and a free end having a tab shape.

7. The head immobilization aid of any one of claims 1-6, wherein the straps, when secured, define an opening between an upper edge of the straps and the rear panel.

8. The head immobilization aid of any one of claims 1-7, wherein at least one of the straps or flaps are constructed of a hook-receptive material.

9. The head immobilization aid of claim 8, wherein the material is FABRIFOAM®.

10. The head immobilization aid of claim 8 or 9, wherein the strap comprises a hook fastener configured to engage the hook-receptive material.

11. The head immobilization aid of any one of claims 8-10, wherein the flap comprises a hook fastener configured to engage the hook-receptive material.

12. The head immobilization aid of any one of claims 1-11, wherein the straps are configured to receive a medical device when in the closed position.

13. A method of immobilizing a head, the method comprising:
(a) placing a head immobilization aid under the head of a patient lying supine or on its side, wherein the head immobilization aid comprises:
(i) a rear panel having lateral edges;
(ii) first and second straps attached to opposing lateral edges of the rear panel;
(iii) two immobilization members spaced laterally apart from each other on opposing lateral edges of the rear panel and attached to said opposing edges; and
(iv) a flap attached to each of the two immobilization members;
(b) engaging a fastener on one strap with the second strap to secure the head immobilization aid to the patient's head, and
(c) engaging a fastener on a flap with a strap to secure the adjacent immobilization aid in a desired position relative to the rear panel, the straps, and the patient's head.

14. The method of claim 13, further comprising repositioning the flaps with respect to the straps to reposition the immobilization members relative to the rear panel, the straps, and the patient's head.

15. The method of claim 14, wherein repositioning the flaps stretches or relaxes the rear panel.

16. The method of any one of claims 13-15, wherein when the fastener on a flap is engaged, the location of engagement of the two straps is covered by the flap.

17. The method of any one of claims 13-16, further comprising securing a medical device to the secured straps.

18. The method of any one of claims 13-17, further comprising securing a medical device to the secured flaps.

19. The method of any one of claims 13-18, wherein when the patient is lying on its side, a rear face of an immobilization member is adjacent the rear face of the other immobilization member.

20. The method of claim 19, wherein the rear panel is slack and securing the flaps secures the immobilization members in relative position to each other.

21. A head immobilization aid for use according to the method of any one of claims 13-20.
